(19) United States
(12) Patent Application Publication
(10) Pub. No.: US 2025/0295898 A1
(43) Pub. Date: Sep. 25, 2025

(54) DEVICE AND METHOD FOR CONFIRMING TISSUE CAPTURE FOR A SHUNT DEVICE

(71) Applicant: EDWARDS LIFESCIENCES CORPORATION, Irvine, CA (US)

(72) Inventors: Morgan Alex Jawitz, Mountain View, CA (US); Sandra Ahide Alcantar Chavez, Anaheim, CA (US); Jeremy Benjamin Scholze, Newport Beach, CA (US)

(73) Assignee: EDWARDS LIFESCIENCES CORPORATION, Irvine, CA (US)

(21) Appl. No.: 19/228,411

(22) Filed: Jun. 4, 2025

Related U.S. Application Data
(63) Continuation of application No. PCT/US2024/010191, filed on Jan. 3, 2024.
(60) Provisional application No. 63/478,879, filed on Jan. 6, 2023.

(51) Int. Cl. A61M 27/00

(52) U.S. Cl. CPC A61M 27/002; A61M 2205/0266; A61M 2205/32; A61M 2210/125

(57) ABSTRACT

A shunt device includes a central passageway, a proximal arm, and a distal arm. The proximal and distal arms are shaped to capture tissue therebetween for anchoring the shunt device. The shunt device preferably includes a spring element having a distal end coupled to the distal arm and a proximal end coupled to the proximal arm. The spring element is stretchable for accommodating the tissue captured between the distal and proximal arms. The spring element is preferably made from a radiopaque material, thereby providing a clinician with a visual indication that tissue has been captured between the proximal and distal arms of the shunt device. A release wire may be coupled to one end of the spring element. The release wire is retractable for releasing the end of the spring element.

### CROSS-REFERENCE TO RELATED APPLICATION(S)

This application is a continuation of International Application No. PCT/US2024/010191, filed Jan. 3, 2024, which claims the benefit of U.S. Provisional Application No. 63/478,879, filed Jan. 6, 2023, the disclosures of which are hereby incorporated by reference in their entireties.

### BACKGROUND

The present disclosure relates generally to implantable devices and more specifically to confirming placement of cardiovascular shunt devices.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt devices can be positioned in the septal wall between the left atrium and the right atrium to shunt blood from the left atrium into the right atrium, thus reducing the pressure in the left atrium.

### SUMMARY

A shunt device has a central flow tube, proximal arm, and distal arm. The proximal arm and distal arm are configured to capture tissue therebetween when implanted in a human body. An apparatus for determining tissue capture of the shunt device includes a coiled spring and first release wire. The coiled spring has a distal end coupled to the distal arm of the shunt device and a proximal end coupled to the proximal arm of the shunt device. The first release wire is coupled to the distal end of the coiled spring. The first release wire is retractable and configured to release the distal end upon retraction of the first release wire. The coiled spring is configured to stretch to accommodate tissue captured between the distal arm and the proximal arm.

A shunt device has a central flow tube, proximal arm, and distal arm. The proximal arm and distal arm are configured to capture tissue therebetween when implanted in a human body. A method of deploying the shunt device includes deploying the distal arm on a first side of a tissue wall, moving the proximal arm toward the distal arm, imaging a coiled spring coupled between the distal arm and the proximal arm, and confirming a state of positioning of the shunt device as being proper positioning in which the coiled spring is in a stretched state indicating tissue capture between the distal arm and the proximal arm or improper positioning in which the coiled spring is in an unstretched state indicating the shunt device has failed to capture tissue between the distal arm and the proximal arm.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

### Anatomy of Heart H and Vasculature V

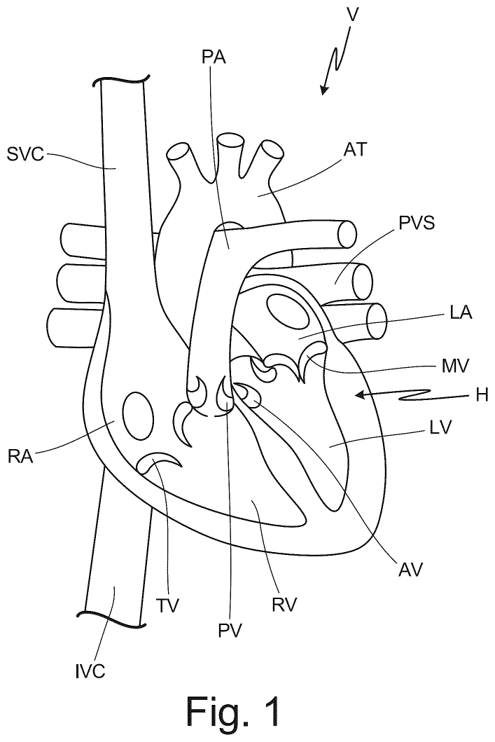

FIG. 1 is a schematic diagram of a heart and vasculature.

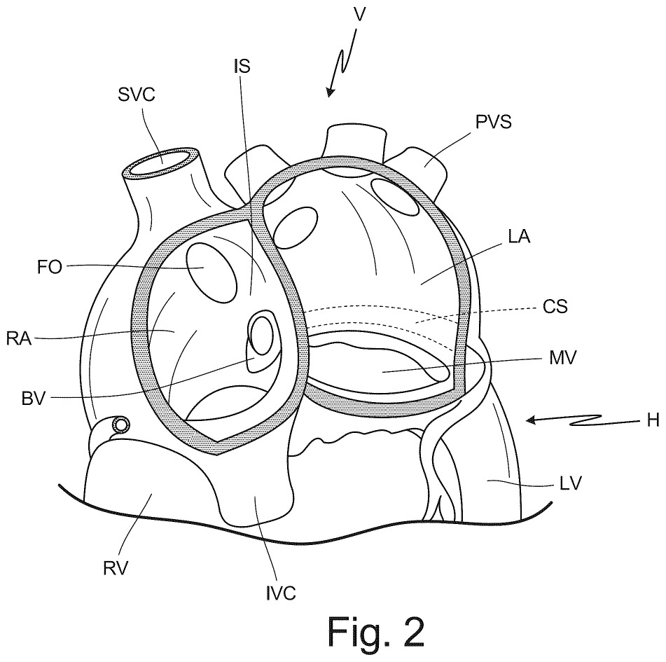

FIG. 2 is a schematic cross-sectional view of the heart.

### Shunt Devices 100 and 100′

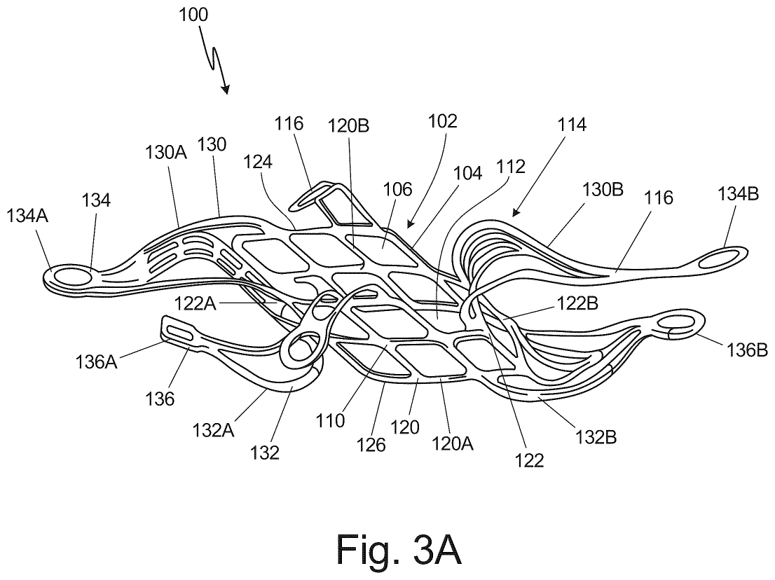

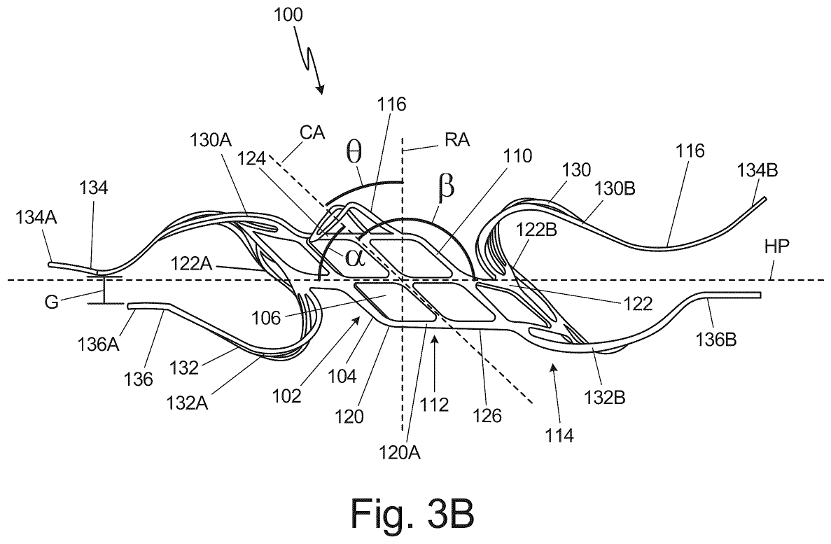

FIG. 3A is a perspective view of a shunt device.

FIG. 3B is a side view of the shunt device.

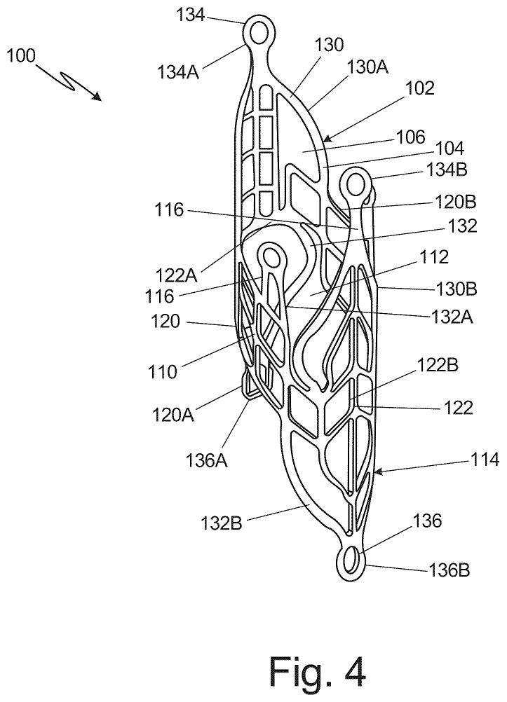

FIG. 4 is a perspective view of the shunt device in a configuration.

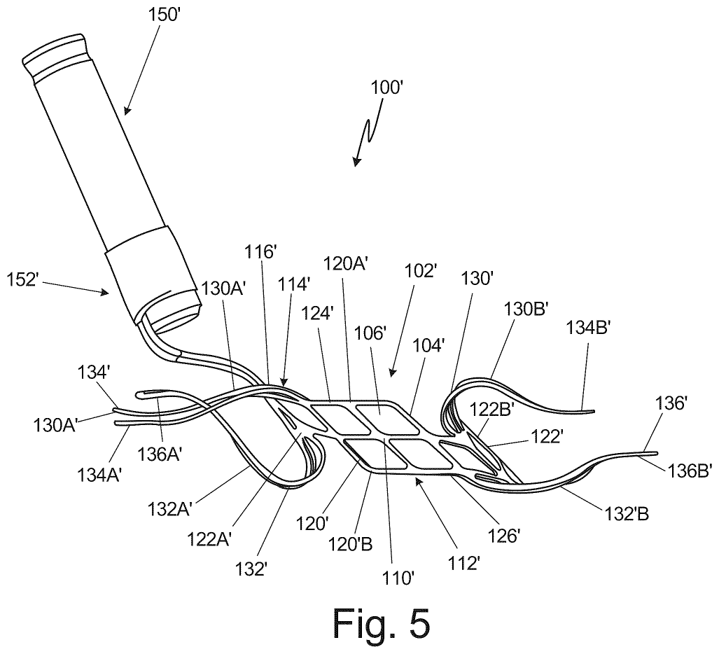

FIG. 5 is a perspective view of a shunt device including a sensor.

### Delivery Catheter 200

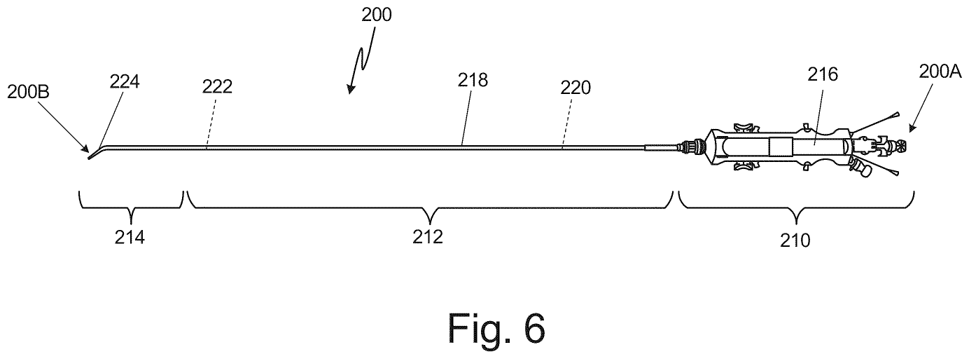

FIG. 6 is a side view of a delivery catheter.

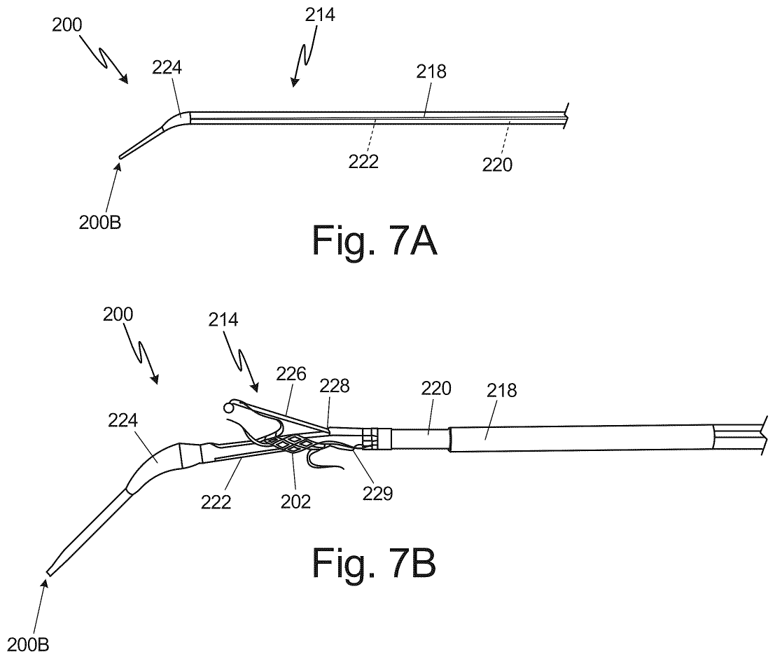

FIG. 7A is a side view of a distal portion of the delivery catheter in a sheathed state.

FIG. 7B is a side view of the distal portion of the delivery catheter in an unsheathed state.

### Delivery Method 300

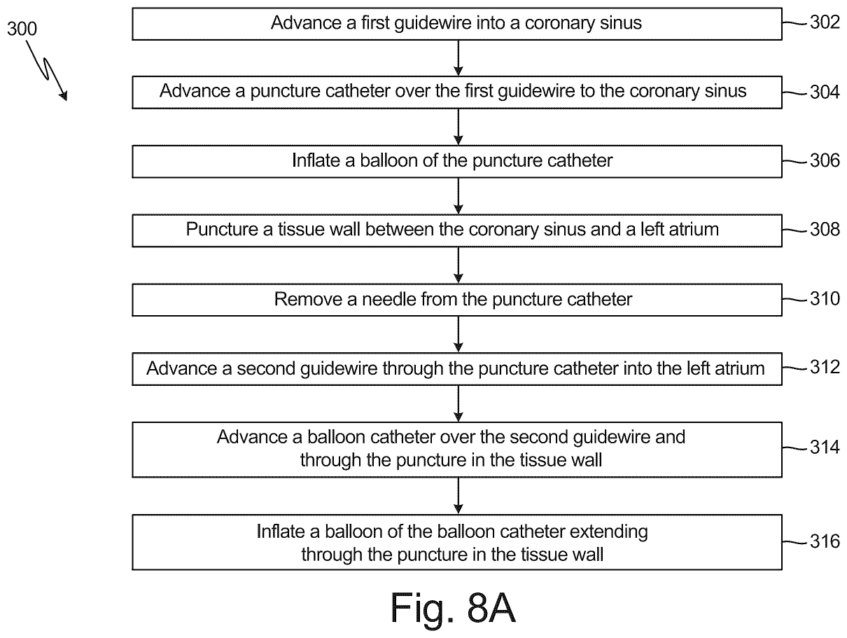

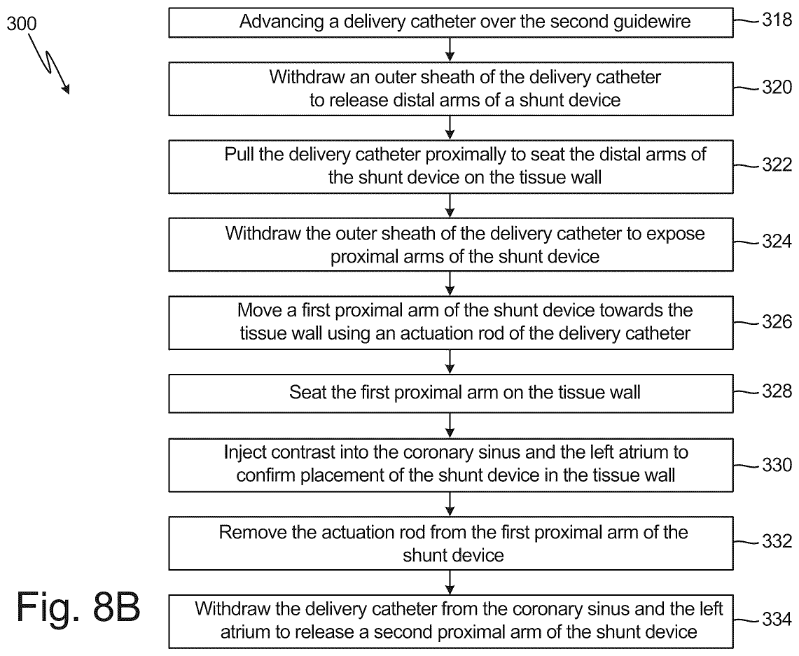

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

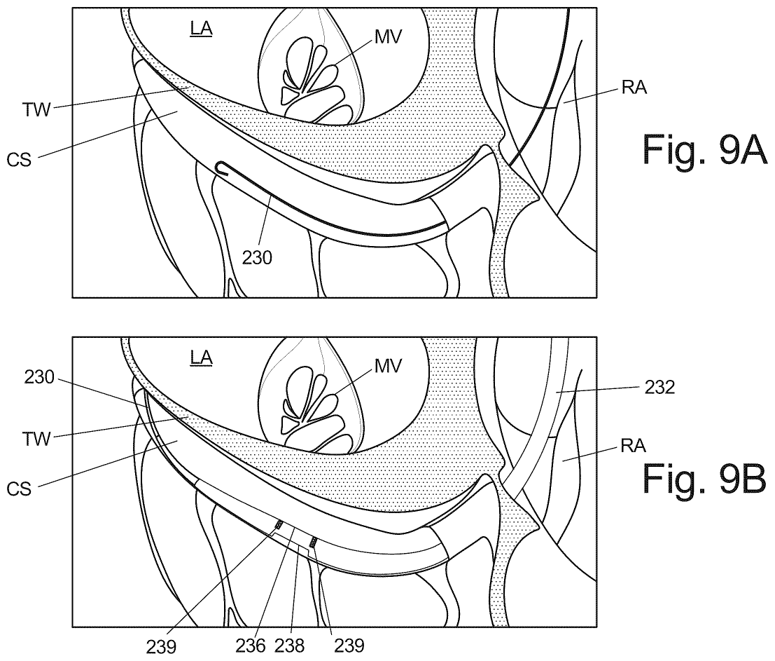

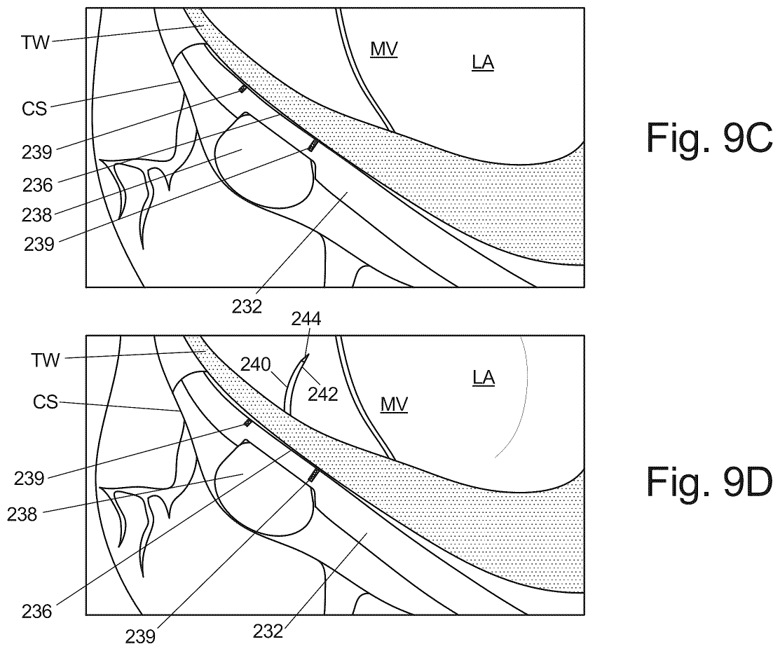

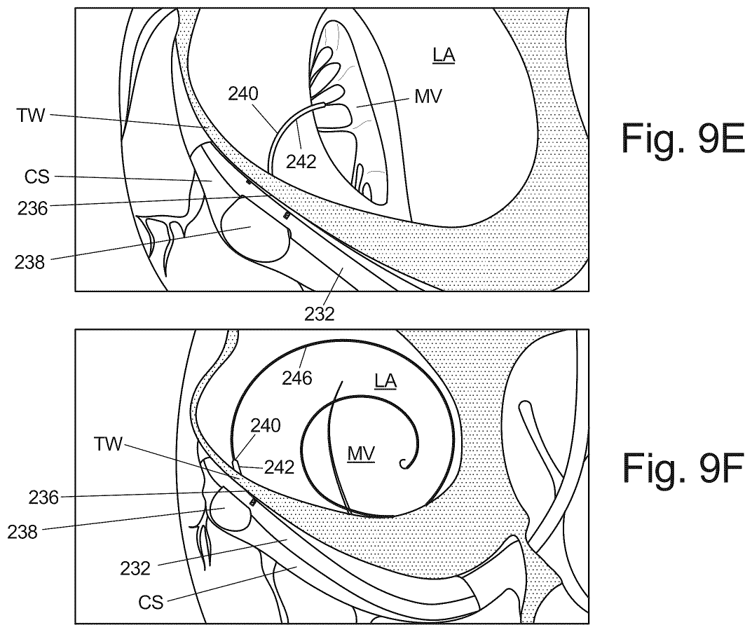

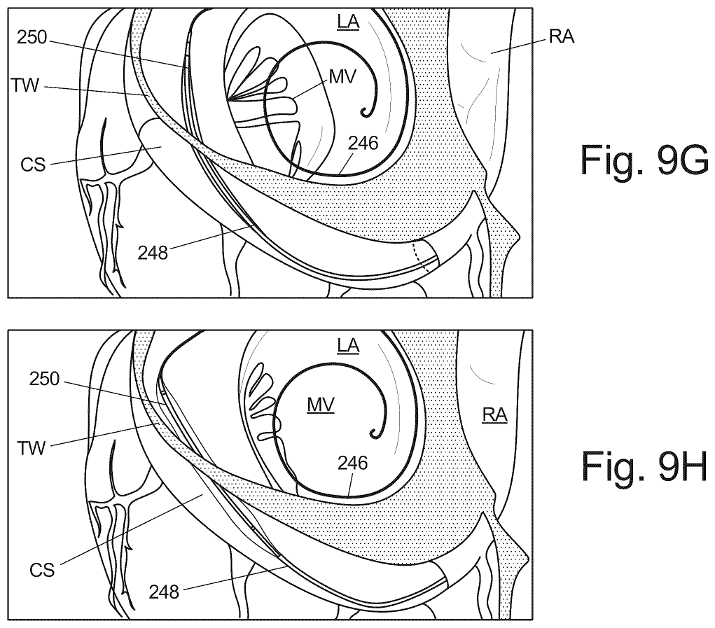

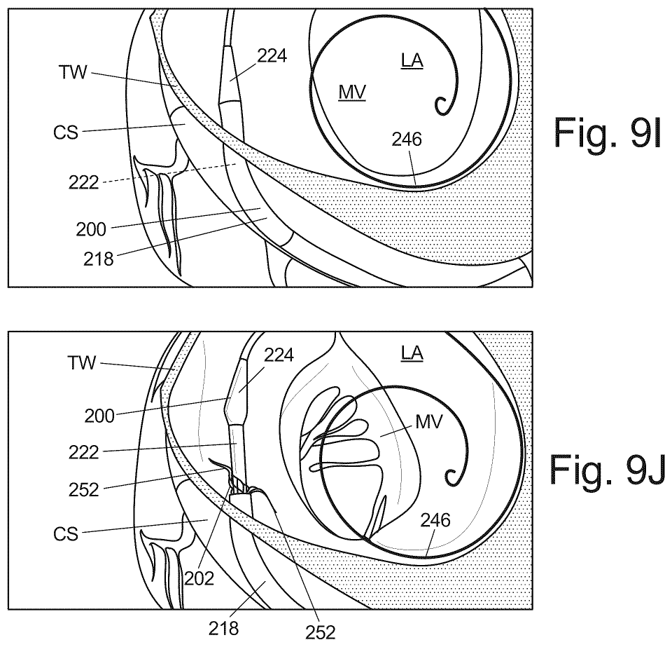

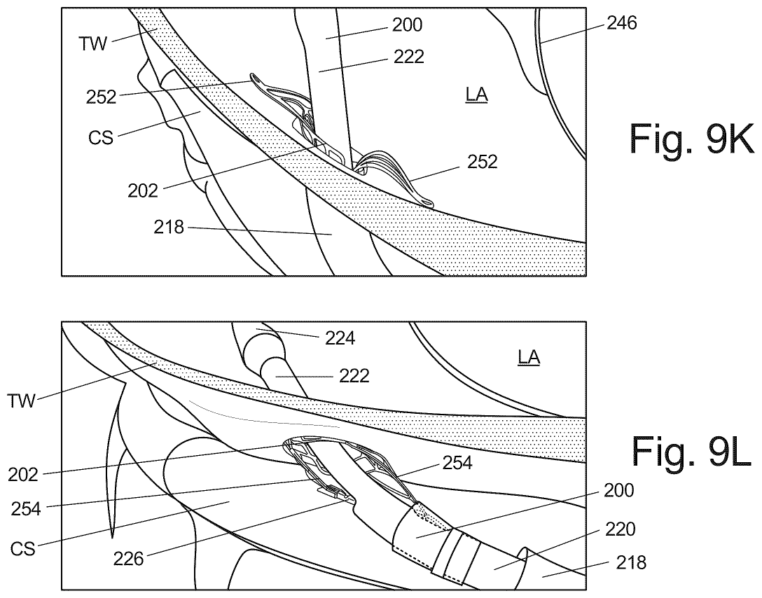

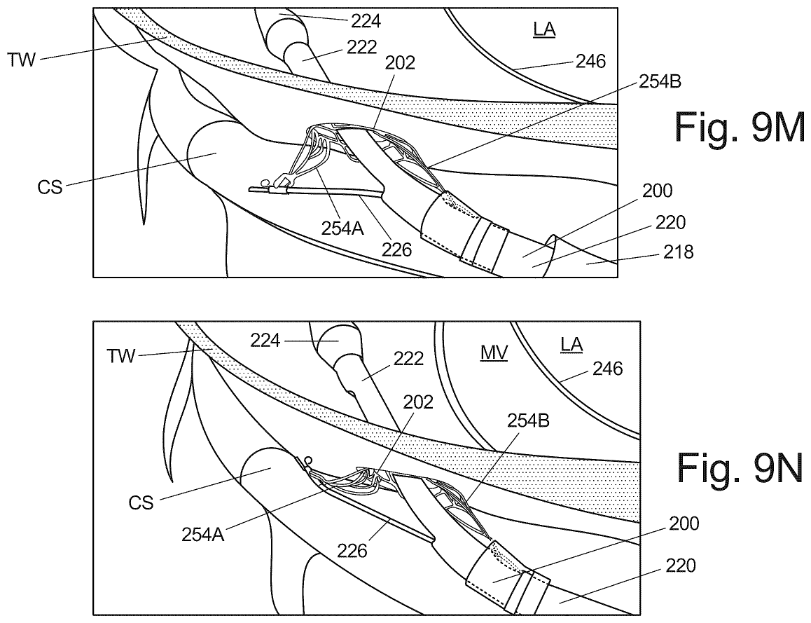

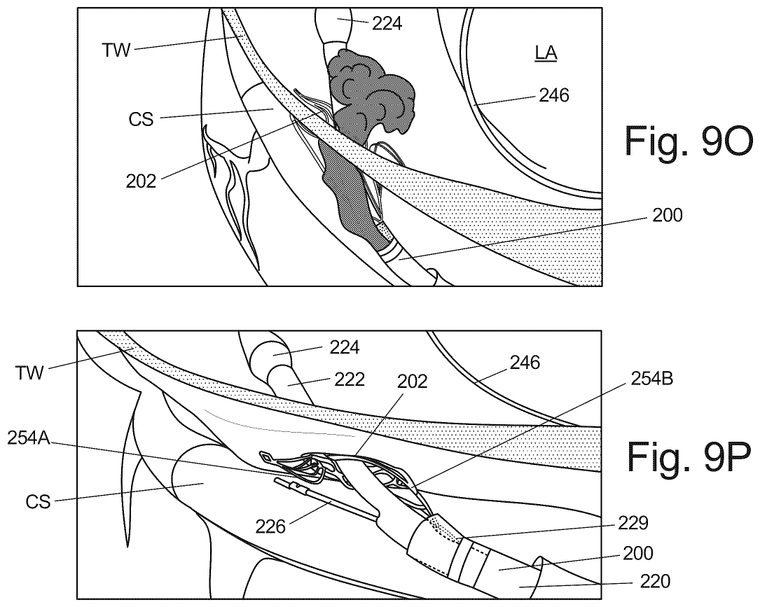

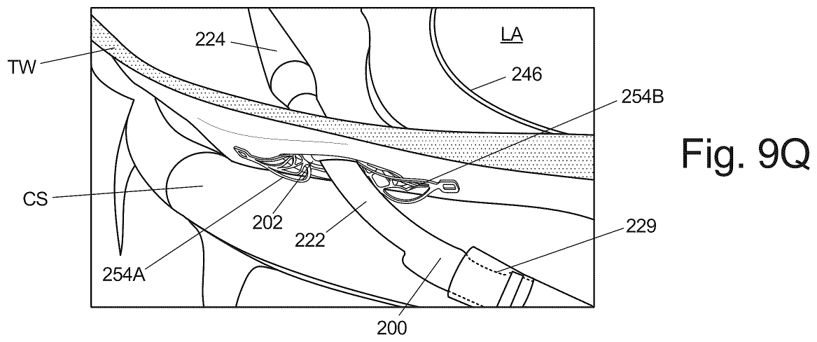

FIGS. 9A-9Q are schematic views showing the steps for implanting a shunt device in the tissue wall between the coronary sinus and the left atrium.

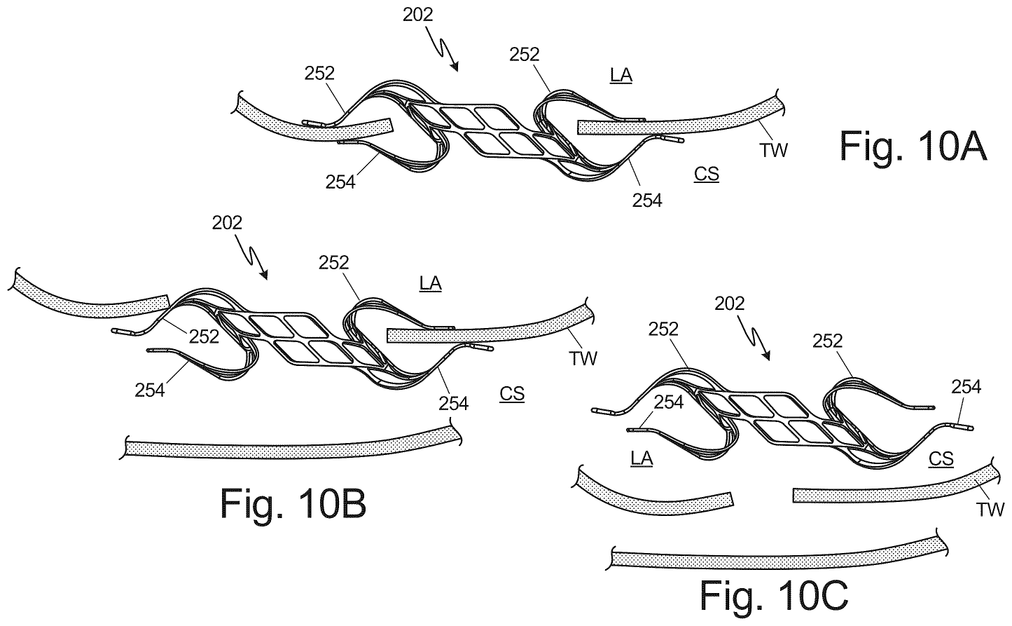

FIG. 10A is a simplified perspective view of the shunt device properly seated between the left atrium and coronary sinus.

FIG. 10B is a simplified perspective view of the shunt device improperly seated between the left atrium and coronary sinus.

FIG. 10C is a simplified perspective view of the shunt device embolized in a left atrium.

### Device and Method for Confirming Tissue Capture

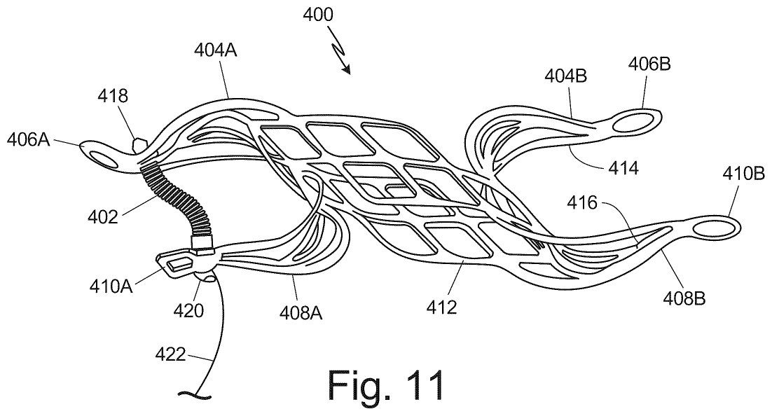

FIG. 11 is a perspective view of a shunt device with a coiled spring for confirming tissue capture.

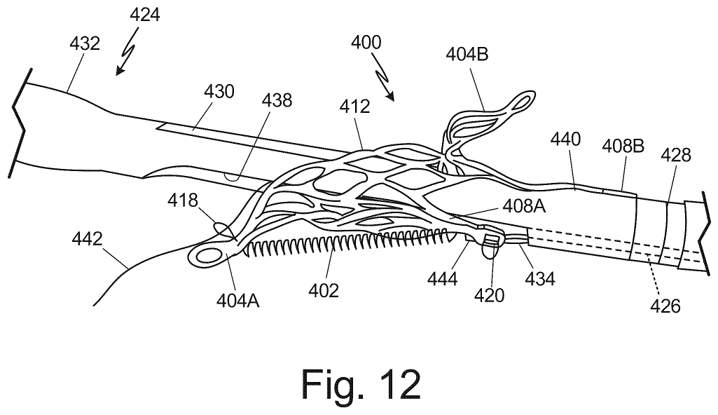

FIG. 12 is a perspective view of a delivery catheter with the shunt device of FIG. 11 having the coiled spring in a stretched state.

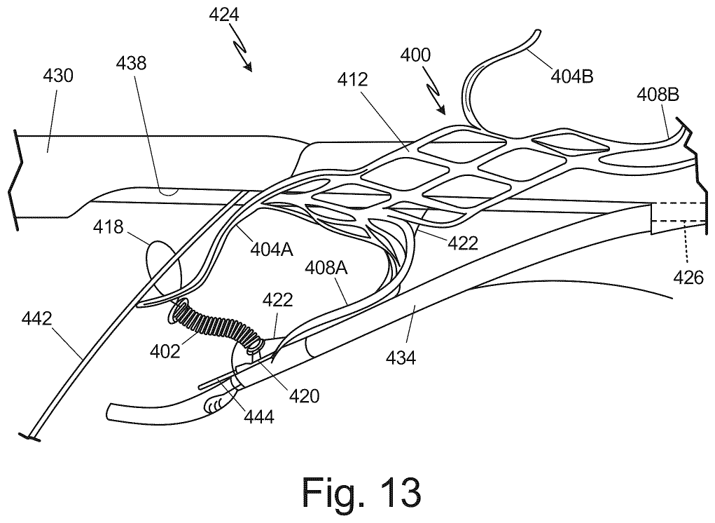

FIG. 13 is a perspective view of the delivery catheter and shunt device of FIG. 12 with the coiled spring in retracted state.

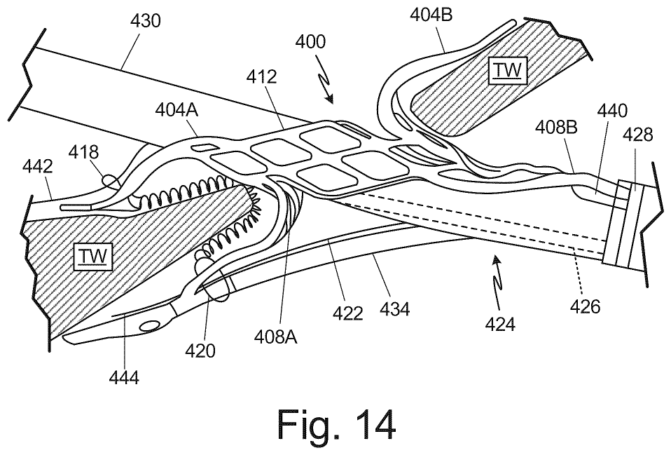

FIG. 14 is a perspective view of the delivery catheter and shunt device of FIG. 12 during deployment of the shunt device between the left atrium and coronary sinus of the human body.

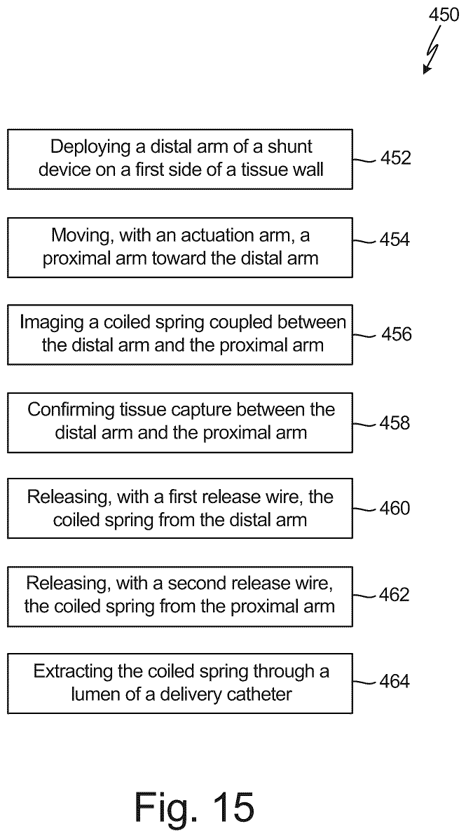

FIG. 15 is a flow chart of a method of deploying the shunt device of FIG. 11 and confirming tissue capture.

While the above-identified figures set forth examples of the present invention, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features, steps and/or components not specifically shown in the drawings.

### DETAILED DESCRIPTION

### Anatomy of Heart H and Vasculature V (FIGS 1-2)

FIG 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees (<90°) and second angle β can be greater than ninety degrees (>90°). In other examples, first angle α and second angle β can be any suitable combination of angles that add to one hundred eighty degrees (180°). The difference between first angle α and second angle β (and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′ (including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9Q.

### Delivery Method 300 (FIGS. 8A-10C)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9Q are schematic views showing the steps for implanting shunt device 202 in tissue wall TW between coronary sinus CS and left atrium LA. FIGS. 8A-9Q will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 9I) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes confirming placement of shunt device 202 in tissue wall TW. FIG. 9O illustrates a known method for confirming tissue confirmation, which includes injecting a contrast agent through a lumen extending through delivery catheter 200. The contrast agent can move through coronary sinus CS and left atrium LA. The contrast will highlight shunt device 202 under fluoroscopy to confirm proper placement of distal arms 252 and first proximal arm 254A of shunt device 202 on tissue wall TW. An alternative method of confirming tissue capture is discussed below.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 10A (below). Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9Q but can be a single catheter in alternate examples.

Shunt devices must be anchored in place to avoid displacement during normal heart rhythms. Techniques are needed to confirmed proper placement of shunt devices during implantation.

FIG. 10A is a simplified perspective view of shunt device 202 properly seated between left atrium LA and coronary sinus CS. FIG. 10B is a simplified perspective view of shunt device 202 improperly seated between left atrium LA and coronary sinus CS. FIG. 10C is a simplified perspective view of shunt device 202 embolized in left atrium LA. FIGS. 10A-10C will be discussed together. FIGS. 10A-10C show shunt device 202, including distal arms 252 and proximal arms 254. FIGS. 10A-10C further show left atrium LA, coronary sinus CS, and tissue wall TW.

FIG. 10A shows shunt device 202 properly seated in tissue wall TW between left atrium LA and coronary sinus CS. As illustrated, distal arms 252 engage tissue wall TW and are positioned in left atrium LA and proximal arms 254 engage tissue wall TW and are positioned in coronary sinus CS. During deployment, one or more distal arms 252 or proximal arms 254 can be mis-seated. In one example, shunt device 202 could be improperly seated such that one or more of distal arms 252 is positioned in coronary sinus CS rather than in left atrium LA, as illustrated in FIG. 10B. For example, during an implantation procedure (e.g., during step 322 of method 300 as shown in FIG. 8B), a physician may pull delivery catheter 200 back too hard after distal arms 252 are released, causing all or a part of shunt device 202 to be pulled into coronary sinus CS. In another example, shunt device 202 could be improperly seated such that the entirety of shunt device 202 is located in left atrium LA, as illustrated in FIG. 10C. For example, during an implantation procedure (e.g., during step 322 of method 300 as shown in FIG. 8B), a physician may not pull delivery catheter 200 back far enough after distal arms 252 are released, so one or more of proximal arms 254 may be released in or pushed through to left atrium LA, causing shunt device 202 to embolize. Confirming tissue capture between the arms of a shunt device helps a physician to determine when it is safe to release the shunt device. As such, confirming proper seating of the shunt device during and/or following delivery also helps reduce the risk of embolization and/or need for redeployment. The present disclosure is directed to an apparatus for determining tissue capture of a shunt device. The disclosed apparatus can be used to confirm the presence of tissue wall TW between distal arms and proximal arms of the shunt device without infusing a contrast agent into the patient. As such, the disclosed apparatus is particularly important for procedures involving patients in which exposure to a contrast agent is contraindicated.

### Device and Method for Confirming Tissue Capture (FIGS. 11-15)

FIG. 11 is a perspective view of a shunt device with a coiled spring for confirming tissue capture. FIG. 11 shows shunt device 400 and coiled spring 402. Shunt device 400 includes distal arms 404A and 404B having terminal ends 406A and 406B, proximal arms 408A and 408B having terminal ends 410A and 410B, central flow tube 412, struts 414, and openings 416. Coiled spring 402 includes distal end 418 and proximal end 420. Coiled spring 402 extends from an uncoiled wire portion 422. Uncoiled wire portion 422 can extend from distal end 418 or proximal end 420.

Shunt device 400 can have the structure and design of any suitable shunt device. Shunt device 400 can be substantially the same as or similar to shunt devices 100, 100′, and 202 illustrated in FIGS. 3A, 3B, 4, 5, 7B, 9J-9Q, and 10A-10C and described with respect thereto. Distal arms 404A and 404B are disposed on opposite sides of central flow tube 412. Proximal arms 408A and 408B are disposed on opposite sides of central flow tube 412. Distal arm 404A and proximal arm 408A are disposed on opposite ends of central flow tube 412 and configured to capture tissue walls TW of left atrium LA and coronary sinus CS therebetween, as shown in FIG. 10A. Distal arm 404B and proximal arm 408B are disposed on opposite ends of central flow tube 412 and configured to capture tissue walls TW of left atrium LA and coronary sinus CS therebetween. Shunt device 400 can be deployed according to method 300 with modification of step 330 relating to tissue capture confirmation as described further herein. Proximal arm 408A corresponds to first proximal arm 254A of shunt device 202 shown, for example in FIG. 9M, moved toward tissue wall with actuation rod 226. Proximal arm 408B corresponds to second proximal arm 245B shown, for example in FIG. 9Q, which is seated on tissue wall TW of coronary sinus CS as delivery catheter 200 is withdrawn.

Shunt device 400 is deployed with coiled spring 402. Coiled spring 402 is used to confirm tissue capture between distal arm 404A and proximal arm 408A. Coiled spring 402 is used in place of delivery of a contrast agent described in step 330 of method 300 and illustrated in FIG. 9O. Coiled spring 402 is disposed between distal arm 404A and proximal arm 408A. Coiled spring 402 includes distal end 418 and proximal end 420. Distal end 418 of coiled spring 402 is coupled to distal arm 404A of shunt device 400. Proximal end 420 of coiled spring 402 is coupled to proximal arm 408A of shunt device 400. As illustrated in FIG. 11, distal end 418 of coiled spring 402 can include a loop, which can be received through an opening (i.e., an opening 416 formed between struts 414) of distal arm 404A of shunt device 400. The opening through which distal end 418 is received can be disposed adjacent to terminal end 406A of distal arm 404A such that coiled spring 402 extends from a location near terminal end 406A of distal arm 404A and substantially separated from central flow tube 412 of shunt device 400. Proximal end 420 of coiled spring 402 can also include a loop, which can be received through an opening (i.e., an opening 416 formed by struts 414) of proximal arm 408A. The opening through which proximal end 420 extends can be disposed adjacent to terminal end 410A of proximal arm 408A such that coiled spring 402 extends from a location near terminal end 410A of proximal arm 408A and substantially separated from central flow tube 412. The separation between coiled spring 402 and central flow tube 412 allows coiled spring 402 to be stretched and displaced by tissue walls TW when tissue walls TW are captured between distal arm 404A and proximal arm 408A of shunt device 400. Coiled spring 402 is formed of a radiopaque material. As described further below, the change in shape of coiled spring 402 can be viewed under fluoroscopy enabling a physician to determine if shunt device 400 is properly positioned with tissue captured between distal arm 404A and proximal arm 408A of shunt device 400. Openings of shunt device 400 through which distal end 418 and proximal end 420 of coiled spring 402 extend can be configured to optimize placement of coiled spring 402 and are not limited to the examples shown in FIGS. 3A and 4. In some examples, one or more struts 414 can define a single, centrally located, opening adjacent to each of terminal ends 406A and 410A. One or more struts 414 can confine distal end 418 and proximal end 420 of coiled spring 402 to a location adjacent to terminal ends 406A and 410A, respectively.

Both shunt device 400 and coiled spring 402 can be formed of a biocompatible shape-memory material, such as nitinol, capable of resuming a preset shape when unconfined. FIG. 11 illustrates shunt device 400 and coiled spring 402 in their preset shapes. As illustrated, coiled spring 402 is in an unstretched state such that the coils of coiled spring 402 are in close proximity (i.e., are not stretched apart). Coiled spring 402 can be formed of a flexible or elastic material that can easily be displaced or stretched by tissue walls TW without damaging tissue walls TW.

Coiled spring 402 can be formed of a radiopaque material (e.g., nitinol) and/or coated with a radiopaque material capable of being viewed under fluoroscopy. Coiled spring 402 can have a diameter thick enough to be visible under fluoroscopy but not thick enough to damage tissue walls TW. In some examples, coiled spring 402 can be formed of a wire having a diameter of approximately 0.008 inches (0.2 millimeters). Coils of coiled spring 402 can have a density and diameter optimized for imaging while minimizing damage to tissue walls TW. The diameter of coils of coiled spring 402 can be limited by a size of a lumen in a delivery catheter through which coiled spring 402 is withdrawn following implantation of shunt device 400. In some examples, coils of coiled spring 402 can have a diameter of approximately 0.032 inches (0.813 millimeters). A length of coiled spring 402 in an unstretched state can be approximately the distance between distal arm 404A and proximal arm 408A of shunt device 400 at the locations where distal end 418 of coiled spring 402 couples to distal arm 404A and proximal end 420 of coiled spring 402 couples to proximal arm 408A. A minimum length of coiled spring in a relaxed or unstretched state can be, for example, approximately 0.82 inches (21 millimeters). Coiled spring 402 in a stretched state can have a length at least equal to a distance between distal arm 404A and proximal arm 408A of shunt device 400 when distal arm 404A and proximal arm 408A are separated in a collapsed configuration on a delivery catheter as described, for example, with respect to FIG. 4.

Uncoiled wire portion 422 is coupled to coiled spring 402. Uncoiled wire portion 422 can be an extension of coiled spring 402. For example, uncoiled wire portion 422 can be an uncoiled portion of coiled spring material extending from distal end 418 or proximal end 420 of coiled spring 402. In some examples, uncoiled wire portion 422 can be an extension of the loop at distal end 418, which can extend through a center of coiled spring 402 and past proximal end 420 of coiled spring 402. In other examples, uncoiled wire portion 422 can be an extension of a loop at proximal end 420. As described further herein, uncoiled wire portion 422 is used to extract coiled spring 402 following delivery of shunt device 400.

FIG. 12 is a perspective view of delivery catheter 424 with shunt device 400 having coiled spring 402 in a stretched state. Delivery catheter 424 is one example of a delivery catheter that can be used to implant shunt device 400 into a patient. Delivery catheter 424 can be substantially the same as or similar to delivery catheter 200 illustrated in FIG. 7B and described with respect thereto. Delivery catheter 424 can take other forms in alternate examples. Lumen 426, inner sheath 428, bridge 430, nosecone 432, actuation arm 434, side opening 438, notch 440, first release wire 442, and second release wire 444 are shown. An outer sheath has been pulled back to release shunt device 400. As described with respect to delivery catheter 200 shown in FIG. 7B, bridge 430 and an outer sheath hold shunt device 400 in a collapsed position on bridge 430 with distal arms 404A and 404B extending away from proximal arms 408A and 408B. As the outer sheath is pulled back, distal arms 404A and 404B return to their preset shape, curving toward proximal arms 408A and 408B, respectively, as shown in FIG. 12.

Proximal arm 408B is secured to bridge 430 at notch 440. Proximal arm 408A is secured to actuation arm 434. Coiled spring 402 is stretched between distal arm 404A and proximal arm 408A. Coiled spring is secured to distal arm 404A by first release wire 442. Coiled spring is secured to proximal arm 408A by second release wire 444. Second release wire 444 can also secure proximal arm 408A to actuation arm 434.

Distal end 418 of coiled spring 402 extends through an opening in distal arm 404A of shunt device 400 and is secured to distal arm 404A by first release wire 442. As shown, a loop at distal end 418 of coiled spring 402 extends through an opening in distal arm 404A of shunt device 400 and is caught by first release wire 442. A distal end of first release wire 442 extends through the loop at distal end 418 of coiled spring 402 to capture and retain distal end 418. The distal end of first release wire 442 can be fed through the loop at distal end 418 of coiled spring 402 during a process of assembling shunt device 400 with delivery catheter 424. In this manner, first release wire 442 can secure distal end 418 of coiled spring 402 to distal arm 404A of shunt device 400 while shunt device 400 is in the collapsed position (e.g., before removal of the outer sheath). First release wire 442 can be formed of a material safe for delivery into the human body. First release wire can have a rigidity and/or diameter sufficient to retain distal end 418 of coiled spring 402 at distal arm 404A of shunt device 400 such that distal end 418 and first release wire 442 are not pulled through distal arm 404A when coiled spring 402 is under tension. A proximal end (not shown) of first release wire 442 can extend through delivery catheter 424 to, for example, handle 216 shown in FIG. 6. The proximal end of first release wire 442 can be configured for manipulation by a user to enable a user to retract first release wire 442. First release wire 442 can be withdrawn proximally through delivery catheter 424 to release distal end 418 of coiled spring 402 upon delivery of shunt device 400.

Proximal arm 408A is secured to actuation arm 434 by second release wire 444, as described in step 332 and illustrated in FIG. 9P. Second release wire 444 can additionally extend through proximal end 420 of coiled spring 402 to secure proximal end 420 to proximal arm 408A. A loop at proximal end 420 of coiled spring 402 can extend through an opening in proximal arm 408A. A distal end of second release wire 444 can extend through the loop at proximal end 420 of coiled spring 402 to capture and retain proximal end 420 and secure proximal end 420 to proximal arm 408A of shunt device 400. Second release wire 444 can be formed of a material safe for delivery into the human body. Second release wire 444 can have a rigidity and/or diameter sufficient to retain proximal arm 408A of shunt device 400 to actuation arm 434 and to secure proximal end 420 of coiled spring 402 to proximal arm 408A such that proximal end 420 and second release wire 444 are not pulled through proximal arm 408A of shunt device 400 when coiled spring 402 is under tension. Second release wire 444 can extend through a lumen (not shown) with actuation arm 434, as described with respect to delivery catheter 200 shown in FIG. 7B. A proximal end (not shown) of second release wire 444 can be located, for example at handle 216 shown in FIG. 6 and configured for manipulation by a user to enable a user to retract second release wire 444. Second release wire 444 can be withdrawn proximally to disconnect second release wire 444 from proximal arm 408A of shunt device 400 and proximal end 420 of coiled spring 402 upon delivery of shunt device 400.

FIG. 12 illustrates the relative arrangement of distal arms 404A and 404B, proximal arms 408A and 408B, and coiled spring 402 after the outer sheath of delivery catheter 424 has been withdrawn. During implantation of shunt device 400, delivery catheter 424 is pulled proximally to seat distal arms 404A and 404B of shunt device 400 on tissue wall TW of left atrium LA, as shown in FIG. 9K and described in step 332 of method 300. During implantation, delivery catheter 424 can be gently pulled proximally to seat distal arms 404A and 404B of shunt device 400 on tissue wall TW in left atrium LA. A physician will stop gently pulling on delivery catheter 424 when resistance is sensed, indicating that distal arms 404A and 404B have come into contact with tissue wall TW. As described further below, during implantation of shunt device 400, coiled spring 402 is displaced by tissue wall TW, which presses against central flow tube 412.

As shown in FIG. 12, the outer sheath of delivery catheter 424 is further withdrawn to expose proximal arms 408A and 408B as described in step 324 of method 300 and shown in FIG. 9L. During implantation, delivery catheter 424 is positioned in left atrium LA, tissue wall TW, and coronary sinus CS so that proximal arms 408A and 408B are positioned in coronary sinus CS when the outer sheath is withdrawn. Proximal arms 408A and 408B are constrained on bridge 430 of delivery catheter 424 and will not automatically assume their preset shape when the outer sheath is withdrawn. As shown, proximal arm 408B is retained in notch 440 and proximal arm 408A is retained on actuation arm 434.

FIG. 13 is a perspective view of delivery catheter 424 and shunt device 400 with coiled spring 402 in a preset unstretched state. Shunt device 400, distal arms 404A and 404B, proximal arms 408A and 408B, central flow tube 412, coiled spring 402, distal end 418, proximal end 420, unoiled wire portion 422, delivery catheter 424, lumen 426, inner sheath 428, bridge 430, actuation arm 434, side opening 438, first release wire 442, and second release wire 444 are shown. FIG. 13 illustrates the relative arrangement of distal arms 404A and 404B, proximal arms 408A and 408B, and coiled spring 402 after proximal arm 408A has been moved into position by actuation arm 434. FIG. 13 illustrates delivery catheter 424 outside of the human body, however the shape of coiled spring 402 illustrated in FIG. 13 also corresponds to the shape of coiled spring 402 upon improper seating of shunt device 400 (e.g., as shown in FIGS. 10B and 10C).

Distal end 418 of coiled spring 402 extends through distal arm 404A of shunt device 400 and is captured and retained by first release wire 442. As shown in FIG. 13, uncoiled wire portion 422 can extend from distal end 418 (e.g., extends from the loop at distal end 418) and can be fed through a center of coiled spring 402 to proximal end 420 of coiled spring 402. Uncoiled wire portion 422 extends through lumen 426. Lumen 426 has a diameter exceeding a diameter of coiled spring 402 to allow coiled spring 402 to be withdrawn through delivery catheter 424 following implantation of shunt device 400.

Proximal end 420 of coiled spring 402 extends through proximal arm 408A of shunt device 400 and is captured and retained by second release wire 444. Second release wire 444 can extend through a loop at proximal end 420 of coiled spring 402 and through an opening in proximal arm 408A of shunt device 400 to retain both coiled spring 402 and shunt device 400 on actuation arm 434.

During implantation of shunt device 400, actuation arm 434 is used to position proximal arm 408A on tissue wall TW in coronary sinus CS. Actuation arm 434 is configured to move proximal arm 408A of shunt device 400 from a first position relative to distal arm 404A of shunt device 400 (shown in FIG. 12) to a second position relative to distal arm 404A (shown in FIG. 13). Coiled spring 402 is in a stretched state when proximal arm 408A of shunt device 400 is in the first position. Proximal arm 408A of shunt device 400 is closer to distal arm 404A of shunt device 400 in the second position. As discussed further below, the shape of coiled spring 402 when proximal arm 408A is in the second position depends on the location of proximal arm 408A and distal arm 404A of shunt device 400 relative to tissue walls TW of left atrium LA and coronary sinus CS. If shunt device 400 is improperly seated, for example, such that both distal arm 404A and proximal arm 408A of shunt device 400 are disposed on the same side of a tissue wall TW of cither left atrium LA or coronary sinus CS and do not capture tissue walls TW, coiled spring 402 will assume a preset unstretched shape. If, however, shunt device 400 is properly seated such that the tissue walls TW of left atrium LA and coronary sinus CS are captured between distal arm 404A and proximal arm 408A of shunt device 400, coiled spring 402 will be in a stretched state as coiled spring 402 is displaced between distal arm 404A and proximal arm 408A by the tissue walls TW.

Coiled spring 402 can be formed of a radiopaque material, e.g., nitinol wire, which can be visible under fluoroscopy. Coiled spring 402 can be configured to be visible in both stretched and unstretched shape. Due to the increased density of coils in an unstretched state, coiled spring 402 may be easier to identify under fluoroscopy in an unstretched state. There are multiple means by which a physician can confirm proper placement of shunt device 400 with the use of coiled spring 402. As discussed further below, during implantation of shunt device 400, the physician can confirm improper positioning of shunt device 400 by viewing coiled spring 402 in an unstretched state under fluoroscopy as illustrated in FIG. 13. The unstretched shape of coiled spring 402 is an indication that shunt device 400 has failed to capture tissue walls TW between distal arm 404A and proximal arm 408A. The physician can confirm proper positioning of shunt device 400 by viewing coiled spring 402 in a stretched state under fluoroscopy when proximal arm 408A is in the second position (i.e., moved via actuation arm 434 toward distal arm 404A). Alternatively, the physician can confirm proper positioning by observing coiled spring 402 transition from an original stretched shape between distal arm 404A and proximal arm 408A when proximal arm 408A is in the first position (collapsed on bridge 430) to modified stretched shape between distal arm 404A and proximal arm 408A when proximal arm 408A is in the second position (i.e., moved by actuation arm 434 toward distal arm 404A). In the original stretched shape, proximal end 420 of coiled spring 402 and proximal arm 408A of shunt device 400 are located at bridge 430 on actuation arm 434 as illustrated in FIG. 12. In the modified stretched shape, proximal end 420 of coiled spring 402 and proximal arm 408A of shunt device 400 are positioned closer to distal arm 404A of shunt device 400 and distal end 418 of coiled spring 402, such that coiled spring 402 has a C-shape (illustrated in FIG. 14). In some examples, coiled spring 402 may only be visible in an unstretched state. As such, a physician may be able to confirm proper positioning of shunt device 400 by confirming the absence of the unstretched coiled spring 402 under fluoroscopy.

FIG. 14 is a perspective view of delivery catheter 424 and shunt device 400 during deployment of the shunt device between left atrium LA and coronary sinus CS of the human body. FIG. 14 shows proximal arm 408A in the second position—moved by actuation arm 434 toward distal arm 404A—consistent with FIG. 13. In contrast to FIG. 13, FIG. 14 shows the shape of coiled spring 402 when tissue walls TW are captured between distal arm 404A and proximal arm 408A of shunt device 400.

Shunt device 400, distal arms 404A and 404B, proximal arms 408A and 408B, central flow tube 412, coiled spring 402, distal end 418, proximal end 420, unoiled wire portion 422, delivery catheter 424, lumen 426, inner sheath 428, bridge 430, actuation arm 434, notch 440, first release wire 442, second release wire 444, left atrium LA, coronary sinus CS, and tissue walls TW are shown. FIG. 14 is intended to show the approximate shape of coiled spring 402 when tissue walls TW are captured between distal arm 404A and proximal arm 408A during implantation of shunt device 400. FIG. 14 is an approximate representation. In practice, tissue walls TW can more completely fill a space between distal arm 404A and proximal arm 408A of shunt device 400 along central flow tube 412.

As shown in FIG. 14, coiled spring 402 can be stretched and displaced by tissue walls TW. Coiled spring 402 can have a C-shape when displaced by tissue walls TW. Coiled spring 402 can be pressed against distal arm 404A, central flow tube 412, and proximal arm 408A of shunt device 400 when displaced by tissue walls TW. Coiled spring can be formed of a material capable of flexing when contacted by tissue walls without causing damage to tissue walls TW. Coiled spring can be, for example, a nitinol wire having a diameter of approximately 0.008 inches (0.2 millimeters), which allows coiled spring 402 to stretch to accommodate tissue walls TW without cutting into or damaging tissue walls TW.

As described with respect to FIGS. 12 and 13, distal end 418 of coiled spring 402 extends through an opening in distal arm 404A of shunt device 400 and is captured by first release wire 442. Proximal end 420 of coiled spring 402 extends through an opening of proximal arm 408A of shunt device 400 and is captured by second release wire 444. Proximal arm 408A of shunt device 400 and proximal end 420 of coiled spring 402 are retained by second release wire 444 on actuation arm 434. Uncoiled wire portion 422 of coiled spring 402 extends from distal end 418 of coiled spring 402 through coiled spring 402 and through lumen 426 of delivery catheter 424.

As previously discussed, depending on the diameter and density of coils of coiled spring 402, coiled spring 402 may or may not be visible under fluoroscopy when displaced by tissue walls TW as shown in FIG. 14. In some examples, the C-shape of coiled spring 402 may be viewed under fluoroscopy, indicating proper positioning of shunt device 400 with tissue capture between distal arm 404A and proximal arm 408A of shunt device 400. In other examples, the absence of coiled spring 402 in an unstretched shape (e.g., as shown in FIG. 13) when viewed under fluoroscopy can indicate proper positioning of shunt device 400.

FIG. 15 is a flow chart of steps of a method 450 for deploying shunt device 400 in a human body and confirming tissue capture.

Step 452 includes deploying distal arms 404A and 404B of shunt device 400 on a first side of tissue wall TW. As described in method 300, an outer sheath of delivery catheter 424 is withdrawn to release distal arms 404A and 404B of shunt device 400. As the outer sheath is withdrawn, distal arms 404A and 404B of shunt device 400 are released and assume their preset shape. If properly positioned, a portion of delivery catheter 424 is located in left atrium LA such that when the outer sheath is withdrawn to release distal arms 404A and 404B of shunt device 400, distal arms 404A and 404B of shunt device 400 are positioned in left atrium LA. Delivery catheter 424 can be pulled proximally to seat distal arms 404A and 404B of shunt device 400 on tissue wall TW in left atrium LA. Proximal arms 408A and 408B are exposed as the outer sheath is further withdrawn. If delivery catheter is properly positioned, proximal arms 408A and 408B are located in coronary sinus CS. Proximal arm 408B is retained on bridge 430 at notch 440. Proximal arm 408A is retained on actuation arm 434.

Step 454 includes moving proximal arm 408A of shunt device 400 from a first position at bridge 430 towards distal arm 404A of shunt device 400 and tissue wall TW of coronary sinus CS. Proximal arm 408A is moved using actuation arm 434 of delivery catheter 424 to a second position as shown in FIG. 14. Actuation arm 434 extends through a lumen in delivery catheter 424 and can be actuated forward to move proximal arm 408A towards distal arm 404A of shunt device 400 and towards tissue wall TW of coronary sinus CS.

Step 456 includes imaging coiled spring 402 coupled between distal arm 404A and proximal arm 408A of shunt device 400 under fluoroscopy. Step 456 can be conducted during and/or after step 454. Coiled spring 402 can be formed from a radiopaque material capable of being viewed under fluoroscopy in one or both of an unstretched state and a stretched state. Coiled spring 402 may be easier to view when unstretched due to the increased density of coils in coiled spring 402 when unstretched. In some examples, coiled spring 402 may not be visible in the stretched state due to the reduced density of coils and small diameter of coiled spring material. In other examples, coiled spring 402 may be visible in both stretched and unstretched states and a change in the shape of coiled spring 402 can be observed as actuation arm 434 moves proximal arm 408A of shunt device 400 toward distal arm 404A of shunt device 400 and toward tissue wall TW of coronary sinus CS.

Step 458 includes confirming tissue capture between distal arm 404A and proximal arm 408A of shunt device 400. Step 458 includes confirming a state of positioning shunt device 400 as being proper positioning in which tissue is captured between distal arm 404A and proximal arm 408A of shunt device 400 or improper positioning in which shunt device 400 fails to capture tissue between distal arm 404A and proximal arm 408A of shunt device 400. When shunt device 400 is properly positioned, coiled spring 402 is in a stretched state, which may or may not be visible under fluoroscopy as described above. When shunt device 400 is improperly positioned, coiled spring 402 is in an unstretched state and is visible under fluoroscopy. When shunt device 400 is improperly positioned, distal arm 404A and proximal arm 408A of shunt device 400 are located on the same side of tissue wall TW in either left atrium LA or coronary sinus CS. When shunt device 400 is properly positioned, distal arm 404A of shunt device 400 is disposed on tissue wall TW in left atrium LA and proximal arm 408A of shunt device 400 is disposed on tissue wall TW in coronary sinus CS.

Coiled spring 402 can be removed upon confirming proper placement of shunt device 400 with tissue walls TW captured between distal arm 404A and proximal arm 408A of shunt device 400. Step 460 includes releasing coiled spring 402 from distal arm 404A. Distal end 418 of coiled spring 402 can be released from distal arm 404A of shunt device 400 by retracting or withdrawing first release wire 442 from distal end 418 of coiled spring 402. A distal end of first release wire 442 can be pulled through a loop at distal end 418 of coiled spring 402 thereby releasing distal end 418 and allowing distal end 418 to be pull through the opening in distal arm 404A of shunt device 400 toward proximal arm 408A of shunt device 400. First release wire 442 can be pulled through a lumen of delivery catheter 424.

Step 462 includes releasing coiled spring 402 from proximal arm 408A of shunt device 400. Proximal end 420 of coiled spring 402 can be released from proximal arm 408A by retracting or withdrawing second release wire 444 from proximal end 420 of coiled spring 402. A distal end of second release wire 444 can be pulled through a loop at proximal end 420 of coiled spring 402 and through an opening in proximal arm 408A of shunt device 400 thereby releasing proximal end 420 of coiled spring 402 and proximal arm 408A of shunt device 400 from actuation arm 434. Second release wire 444 can be pulled through a lumen with actuation arm 434. Coiled spring 402 is free from shunt device 400 following release of distal end 418 and proximal end 420 of coiled spring 402.

Step 464 includes extracting coiled spring 402 from left atrium LA and coronary sinus CS through lumen 426 of delivery catheter 424. Coiled spring 402 is used for tissue capture confirmation and is not required once a physician has determined that shunt device 400 has been properly positioned. Coiled spring 402 can be withdrawn through lumen 426 by pulling uncoiled wire portion 422, which extends through lumen 426. Uncoiled wire portion 422 and coiled spring 402 can be pulled into lumen 426 of delivery catheter 424. Lumen 426 is sized to accommodate coiled spring 402.

Before or after coiled spring 402 has been removed, proximal arm 408B can be released. After shunt device has been properly seated between tissue walls TW of left atrium LA and coronary sinus CS, delivery catheter 424 can be removed as described in step 334 of method 300 above.

The use of coiled spring 402 as described herein can provide a reliable means for confirming tissue capture of a shunt device without the use of a contrast agent. Coiled spring 402 can be particularly beneficial for use in patients for which exposure to contrast is contraindicated. Coiled spring 402 can formed of a radiopaque flexible material that can be visible under fluoroscopy and deployed and extracted without damaging tissue walls TW.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter or value, without variation, as if no qualifying relative term or term of degree were utilized in the given disclosure or recitation.

### DISCUSSION OF DETAILED EMBODIMENTS

The following are non-exclusive descriptions of possible embodiments of the present invention.

A shunt device has a central flow tube, proximal arm, and distal arm. The proximal arm and distal arm are configured to capture tissue therebetween when implanted in a human body. An apparatus for determining tissue capture of the shunt device includes a coiled spring and first release wire. The coiled spring has a distal end coupled to the distal arm of the shunt device and a proximal end coupled to the proximal arm of the shunt device. The first release wire is coupled to the distal end of the coiled spring. The first release wire is retractable and configured to release the distal end upon retraction of the first release wire. The coiled spring is configured to stretch to accommodate tissue captured between the distal arm and the proximal arm.

The apparatus of the preceding paragraph can optionally include, additionally and/or alternatively, any one or more of the following features, configurations and/or additional components:

A further embodiment of the apparatus of the foregoing paragraph, wherein the distal end of the coiled spring can include a loop.

A further embodiment of the apparatus of any of the foregoing paragraphs, wherein the loop can be received through an opening of the distal arm and wherein the loop is captured by the first release wire to secure the distal end of the coiled spring to the distal arm. A first end of the first release wire can extend through the loop.

A further embodiment of the apparatus of any of the foregoing paragraphs, wherein a second end, opposite the first end of the first release wire, can be configured to extend through a delivery catheter.

A further embodiment of the apparatus of any of the foregoing paragraphs, wherein the coiled spring can be configured to maintain an unstretched shape when the shunt device is improperly positioned such that the shunt device fails to capture tissue between the distal arm and the proximal arm.

A further embodiment of the apparatus of any of the foregoing paragraphs can further include an actuation rod coupled to the proximal arm and configured to position the proximal arm during deployment of the shunt device.

A further embodiment of the apparatus of any of the foregoing paragraphs, wherein the actuation rod can include a second release wire. The second release wire can be configured to retain the proximal arm and the proximal end of the coiled spring.

A further embodiment of the apparatus of any of the foregoing paragraphs, wherein the second release wire can be retractable and configured to release the proximal arm and the proximal end of the coiled spring upon retraction of the second release wire.

A further embodiment of the apparatus of any of the foregoing paragraphs, wherein the proximal end of the coiled spring can include a loop and wherein the second release wire can extend through the loop to capture the proximal end of the coiled spring.

A further embodiment of the apparatus of any of the foregoing paragraphs, wherein the actuation arm can be configured to move the proximal arm from a first position relative to the distal arm to a second position relative to the distal arm, wherein the proximal arm is closer to the distal arm in the second position.

A further embodiment of the apparatus of any of the foregoing paragraphs, wherein the coiled spring can be in a stretched state when the proximal arm is in the first position.

A further embodiment of the apparatus of any of the foregoing paragraphs, wherein the coiled spring can be in an unstretched state when the proximal arm is in the second position and the proximal arm and the distal arm are disposed on the same side of a tissue wall such that tissue is not captured between the proximal arm and the distal arm.

A further embodiment of the apparatus of any of the foregoing paragraphs, wherein the coiled spring can be in a stretched state when the proximal arm is in the second position and tissue is captured between the proximal arm and the distal arm.

A further embodiment of the apparatus of any of the foregoing paragraphs, wherein the proximal end of the coiled spring can extend from an uncoiled wire portion, the uncoiled wire portion configured to be pulled to remove the coiled spring from the shunt device following deployment of the shunt device.

A further embodiment of the apparatus of any of the foregoing paragraphs, wherein the uncoiled wire portion can extend through a lumen of a delivery catheter, wherein the lumen is configured to receive the coiled spring following removal of the coiled spring from the shunt device.

A further embodiment of the apparatus of any of the foregoing paragraphs, wherein the coiled spring can be radiopaque.

A further embodiment of the apparatus of any of the foregoing paragraphs, wherein the coiled spring can be a nitinol wire.

A further embodiment of the apparatus of any of the foregoing paragraphs, wherein the distal end of the coiled spring can be coupled to the distal arm adjacent to a terminal end of the distal arm and the proximal end of the coiled spring can be coupled to the proximal arm adjacent to a terminal end of the proximal arm.

A shunt device has a central flow tube, proximal arm, and distal arm. The proximal arm and distal arm are configured to capture tissue therebetween when implanted in a human body.

A method of deploying the shunt device includes deploying the distal arm on a first side of a tissue wall, moving the proximal arm toward the distal arm, imaging a coiled spring coupled between the distal arm and the proximal arm, and confirming a state of positioning of the shunt device as being proper positioning in which the coiled spring is in a stretched state indicating tissue capture between the distal arm and the proximal arm or improper positioning in which the coiled spring is in an unstretched state indicating the shunt device has failed to capture tissue between the distal arm and the proximal arm.

The method of the preceding paragraph can optionally include, additionally and/or alternatively, any one or more of the following features, configurations, additional components, and/or steps:

A further embodiment of the method of the foregoing paragraph can further include, upon confirming proper positioning of the shunt device, the steps of releasing the coiled spring from the distal arm, releasing the coiled spring from the proximal arm, and extracting the coiled spring from the shunt device through a lumen of a delivery catheter.

A further embodiment of the method of the foregoing paragraph, wherein releasing the coiled spring from the distal arm can include retracting a first release wire coupling a distal end of the coiled spring to the distal arm, and wherein releasing the coiled spring from the proximal arm can include retracting a second release wire coupling a proximal end of the coiled spring to the proximal arm.

The above method(s) can be performed on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. An apparatus for determining tissue capture of a shunt device, the shunt device having a central flow tube, a proximal arm, and a distal arm, the proximal arm and the distal arm configured to capture tissue therebetween when implanted in a human body, the apparatus comprising:
a coiled spring having a distal end coupled to the distal arm of the shunt device and a proximal end coupled to the proximal arm of the shunt device;
wherein the coiled spring is configured to stretch to accommodate tissue captured between the distal arm and the proximal arm.

2. The apparatus of claim 1 and further comprising a first release wire coupled to the distal end of the coiled spring, wherein the first release wire is retractable and configured to release the distal end upon retraction of the first release wire.

3. The apparatus of claim 2 and further comprising an actuation arm coupled to the proximal arm and configured to position the proximal arm during deployment of the shunt device.

4. The apparatus of claim 3, wherein the actuation arm comprises a second release wire, wherein the second release wire is configured to retain the proximal arm and the proximal end of the coiled spring.

5. The apparatus of claim 4, wherein the second release wire is retractable and configured to release the proximal arm and the proximal end of the coiled spring upon retraction of the second release wire.

6. The apparatus of claim 5, wherein the proximal end of the coiled spring comprises a loop and wherein the second release wire extends through the loop to capture the proximal end of the coiled spring.

7. The apparatus of claim 3, wherein the actuation arm is configured to move the proximal arm from a first position relative to the distal arm to a second position relative to the distal arm, wherein the proximal arm is closer to the distal arm in the second position.

8. The apparatus of claim 7, wherein the coiled spring is in a stretched state when the proximal arm is in the first position.

9. The apparatus of claim 7, wherein the coiled spring is in an unstretched state when the proximal arm is in the second position and the proximal arm and the distal arm are disposed on the same side of a tissue wall such that tissue is not captured between the proximal arm and the distal arm, and wherein the coiled spring is in a stretched state when the proximal arm is in the second position and tissue is captured between the proximal arm and the distal arm.

10. The apparatus of claim 1, wherein the proximal end of the coiled spring extends from an uncoiled wire portion, the uncoiled wire portion configured to be pulled to remove the coiled spring from the shunt device following deployment of the shunt device.

11. The apparatus of claim 10, wherein the uncoiled wire portion extends through a lumen of a delivery catheter, wherein the lumen is configured to receive the coiled spring following removal of the coiled spring from the shunt device.

12. An apparatus for determining tissue capture of a shunt device, the shunt device having a central flow tube, a proximal arm, and a distal arm, the proximal arm and the distal arm configured to capture tissue therebetween when implanted in a human body, the apparatus comprising:
a spring element releasably coupled to each of the distal arm of the shunt device and the proximal arm of the shunt device;
wherein the spring element is releasably coupled to the distal arm and the proximal arm adjacent to terminal ends of each of the distal arm and the proximal arm; and
wherein the spring element is configured to stretch to accommodate tissue captured between the distal arm and the proximal arm.

13. The apparatus of claim 12 and further comprising a first release wire coupled to a distal end of the spring element, wherein the first release wire is retractable and configured to release the distal end upon retraction of the first release wire.

14. The apparatus of claim 13, wherein the spring element comprises a coiled wire and wherein distal end of the spring element comprises a loop, the loop received through an opening of the distal arm and captured by the first release wire to secure the distal end of the spring element to the distal arm, wherein a first end of the first release wire extends through the loop.

15. The apparatus of claim 14, wherein a second end of the first release wire, opposite the first end of the first release wire, is configured to extend through a delivery catheter.

16. A shunt device comprising:
a central flow tube;
a proximal arm;
a distal arm, wherein the proximal arm and the distal arm are configured to capture tissue therebetween when implanted in a human body; and
a spring element having a distal end removably coupled to the distal arm of the shunt device and a proximal end removably coupled to the proximal arm of the shunt device;
wherein the spring element is configured to stretch to accommodate tissue captured between the distal arm and the proximal arm and to resume a preset unstretched state when unconfined.

17. The shunt device of claim 16, wherein the spring element is radiopaque.

18. The shunt device of claim 17, wherein the spring element comprises a coiled nitinol wire.

19. The shunt device of claim 16, wherein the spring element comprises a coiled wire and wherein the distal end of the spring element comprises a loop.

20. The shunt device of claim 19, wherein the loop is received through an opening of the distal arm and wherein the loop is configured to receive a release wire to secure the distal end of the spring clement to the distal arm.
